(19) United States
(12) Patent Application Publication
(10) Pub. No.: US 2013/0041326 A1
(43) Pub. Date: Feb. 14, 2013

(54) INJECTION APPARATUS FOR LONG DISTANCE DELIVERY OF SOFT TISSUE BULKING AGENTS CONTAINING MICROSPHERES

(71) Applicants: Corbett W. Stone, San Diego, CA (US); Stefan M. Lemperle, La Jolla, CA (US); Russell J. Anderson, San Diego, CA (US); Gottfried H. Lemperle, La Jolla, CA (US)

(72) Inventors: Corbett W. Stone, San Diego, CA (US); Stefan M. Lemperle, La Jolla, CA (US); Russell J. Anderson, San Diego, CA (US); Gottfried H. Lemperle, La Jolla, CA (US)

(73) Assignee: ASCENTX MEDICAL, INC., Dana Point, CA (US)

(21) Appl. No.: 13/641,671

(22) PCT Filed: Apr. 15, 2011
(86) PCT No.: PCT/US11/32730

§ 371 Date: Oct. 16, 2012

Related U.S. Application Data
(60) Provisional application No. 61/325,138, filed on Apr. 16, 2010.

(51) Int. Cl. A61M 5/315

(52) U.S. Cl. 604/218

(57) ABSTRACT

An injection device is formed as a long tubular structure with a wire that controls a piston in the structure from a proximal end, allowing therapeutic material to be injected into a patient through a needle at a distal end.

### CROSS-REFERENCE TO RELATED APPLICATIONS

This application claims the benefit of Prov. App. 61/325,138 (Att. Docket AX8379PR), filed Apr. 16, 2010 and entitled SYRINGE FOR LONG DISTANCE DELIVERY OF MEDICAMENTS, the entire contents of which are hereby incorporated by reference.

### BACKGROUND OF THE INVENTION

1. Field of the Invention

The present invention relates generally to medical devices and, more particularly, to syringes and needles.

2. Description of Related Art

Medical procedures often require delivery of a fluid or solid suspension to a location that is difficult to reach in the body of a patient. Examples of such difficult locations include the lower esophageal sphincter (LES), located at the lower end of the esophagus, the urinary sphincter, which is located at the urinary outflow of the bladder into the urethra and a relatively long distance from the urinary meatus, particularly so in males, and the internal anal sphincter. The LES, for example, is an anatomic structure that, as the patient gets older, may lose the ability to stay completely closed over a long period of time due to an insufficient sphincter tonus. Consequently, stomach acid may leak up into the esophagus leading to gastroesophageal reflux disease (GERD), which often may be labeled “heartburn” and which can be very serious and painful, and even lead to esophageal cancer. Worldwide, an estimated 75 million people suffer from this condition on a regular (e.g., daily) basis.

During proper operation of the lower esophageal sphincter, the lower esophageal sphincter opens to allow food to pass into the stomach and doses to prevent food and acidic stomach fluids from flowing back up into the esophagus. Gastroesophageal reflux occurs when the lower esophageal sphincter is weak or relaxes inappropriately, allowing the stomach's contents to retrograde or flow up into the esophagus. This retrograde flow of gastric contents back into the esophagus, through what should be a one-way valve into the stomach, can damage the esophagus. More particularly, the contents of the stomach are very acidic, and only the lining of the stomach is specifically designed to cope with the lower pH contents. The esophagus, on the other hand, is not suited for such exposure to highly acidic materials. Thus, when acid retrogrades from the stomach into the esophageal tissues, irritation and inflammation will often result to these tissues.

The severity of tissue damage, which results from gastroesophageal reflux disease, depends on factors such as intermittent sphincter relaxation and lack of sufficient sphincter pressure (tone), as well as the composition and amount of fluid refluxed or regurgitated backwards from the stomach. Another factor, which may affect the severity of a particular gastroesophageal reflux disorder, is the patient's esophageal motility. Lack of esophageal motility can occur through either of two mechanisms. When incomplete emptying of the esophagus into the stomach after ingestion of liquids or solids occurs, the motility of the esophagus can be said to be effected, resulting in esophageal reflux. Also, esophageal reflux can occur when small amounts of gastric contents, which may be refluxed into the lower esophagus, are not rapidly emptied back into the stomach. Delays in the emptying of this material, caused by an esophageal motility disorder, for example, can lead to irritation of the esophageal mucosa and possibly to the sensation of heartburn or the development of esophagitis.

Treatment of GERD may involve surgery. One surgical procedure, known as Nissen fundoplication, is considered to be the gold standard of surgical procedures addressing GERD and is today considered one of the most effective.

With regard to the urinary sphincter, the term “stress urinary incontinence” is caused by a functionally insufficient urinary sphincter muscle of a patient. In a patient having this condition, an insufficient urinary sphincter tonus at the urinary outflow of the bladder into the urethra can cause a loss of bladder control. Cystoscopes are typically used to study the urethra and bladder and to evaluate a patient's urinary incontinence condition. A typical cystoscope may comprise a tubular instrument equipped with, for example, a visual channel and a working channel, and constructed to be inserted through the urethra for viewing of the urethra and bladder.

Various tools and instruments have been used in the prior art for the treatment of types of conditions such as the above-mentioned acid reflux disease and urinary incontinence. Gastroscopes are typically used to study the esophagus and to evaluate, for example, a patient's acid reflux condition. A gastroscope typically comprises a flexible, lighted instrument that is inserted through the mouth and esophagus to view the stomach. Similarly, a cystoscope is typically inserted through a patient's urethra to facilitate evaluation of for example, a urinary incontinence condition.

A material having relatively high viscosity, such as collagen (and/or a material such hyaluronic acid (HA)), may be injected into the vicinity of either the lower esophageal sphincter (for GERD) or the sphincter of the urethra (for urinary incontinence) to treat either of these disorders by ‘bulking’ surrounding soft tissues and thereby increasing and re-establishing the sphincter pressure. Injection procedures typically involve elongated catheters for delivery of therapeutic materials through body passages to target sites of injection. The force required to deliver a highly viscous material through a delivery lumen of an elongated catheter increases as the average viscosity of the material being delivered increases and as the length of the elongated catheter increases.

A need thus exists in the prior art for an instrument for injecting medicaments precisely and predictably into certain anatomical structures in relatively distant locations.

### SUMMARY OF THE INVENTION

The invention herein disclosed comprises, according to one embodiment, an elongated body having disposed therein a movable piston having a distal side and a proximal side, wherein the piston divides the body into a first portion proximal to the piston and a second portion distal to the piston. The embodiment further comprises a wire having a distal end attached to the proximal side of the piston, the wire extending to a proximal end of the first body portion and being controllable from the proximal end of the first body portion, thereby making the piston capable of being moved by the wire within the elongated body. Further, a hollow distal needle is disposed at a distal end of the second body portion, whereby, when the distal needle is inserted into the body of a patient, motion of the piston is capable of causing material disposed in an interior of the second body portion to be injected into the body of the patient.

Another embodiment of the present invention may further comprise a tissue stop disposed around the distal needle no that the distal needle is inserted only up to a predetermined depth at the injection site.

While the apparatus and method have or will be described for the sake of grammatical fluidity with functional explanations, it is to be expressly understood that the claims, unless expressly formulated, are not to be construed as necessarily limited in any way by the construction of “means” or “steps” limitations, but are to be accorded the full scope of the meaning and equivalents of the definition provided by the claims under the doctrine of equivalents, and in the case where the claims are expressly formulated are to be accorded full statutory equivalents.

Any feature or combination of features described herein are included within the scope of the present invention provided that the features included an such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a simplified cross-sectional drawing of proximal and distal portions of a long injection device according to the present invention;

FIG. 1A is a cross-sectional drawing of a portion of a long injection device employing a wire-activated piston;

FIG. 1B is a cross-sectional diagram illustrating disposition of a tissue stop around a needle of the long injection device;

FIG. 2 is a cross-sectional drawing showing detail of the distal portion of the long injection device of FIG. 1;

FIG. 3A is a cross-sectional sketch of an embodiment of a crab washer mechanism in a locked state;

FIG. 3B illustrates, in cross-section, an embodiment of a crab washer mechanism in an unlocked state;

FIG. 4 is an illustration of an embodiment of a ratchet mechanism for advancing a wire in a long injection device;

FIG. 5 is an illustration of an embodiment of a portion of double roller system for advancing a wire in a long injection device;

FIG. 6A is a schematic diagram describing attachment of a wire to a piston in a long injection device;

FIG. 6B is an illustration of a single head implementation of a distal end of a wire onto which a piston may be molded;

FIG. 6C is a sketch of a double head of an exemplary embodiment of a piston disposed at the distal end of a wire in a long injection device;

FIG. 7 is a perspective view of a vial affixed to a pistol grip for injecting therapeutic material using a long injection device; and

FIG. 8 depicts a low cost needle with minimal flow losses and a construction free of ridge(s) that if present could allow microspheres to collect.

### DETAILED DESCRIPTION OF THE PRESENT INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may full within the spirit and scope of the invention as defined by the appended claims. The present invention may be practiced in conjunction with various injection devices that are conventionally used in the art. For purposes of illustration, the present invention may be adapted to an injection device incorporating a medical injection or injection facilitation apparatus, e.g., a transition-bore needle apparatus, as disclosed in U.S. Pat. No. 6,666,848 (the 848 patent). As another example, an elongated or elongated flexible syringe as described in U.S. Pat. No. 6,929,623 (the '623 patent) may be modified to include aspects of the present invention. The present invention, further, may be adapted to structures and/or methods described in “Endoscopic lower esophageal sphincter bulking for the treatment of GERD: safety evaluation of injectable polymethylmethacrylate microspheres in miniature swine,” by Jan P. Kamler, et al, and published in Gastrointestinal Endoscopy, Volume 72, No. 2: 2010, cited below. The contents of the '848 patent, the '623 patent and the Kanter article are incorporated herein by reference in their entireties with respect to the methods and/or structures described therein for adaptation in any combination or permutation with the disclosure set forth herein to the extent not mutually exclusive.

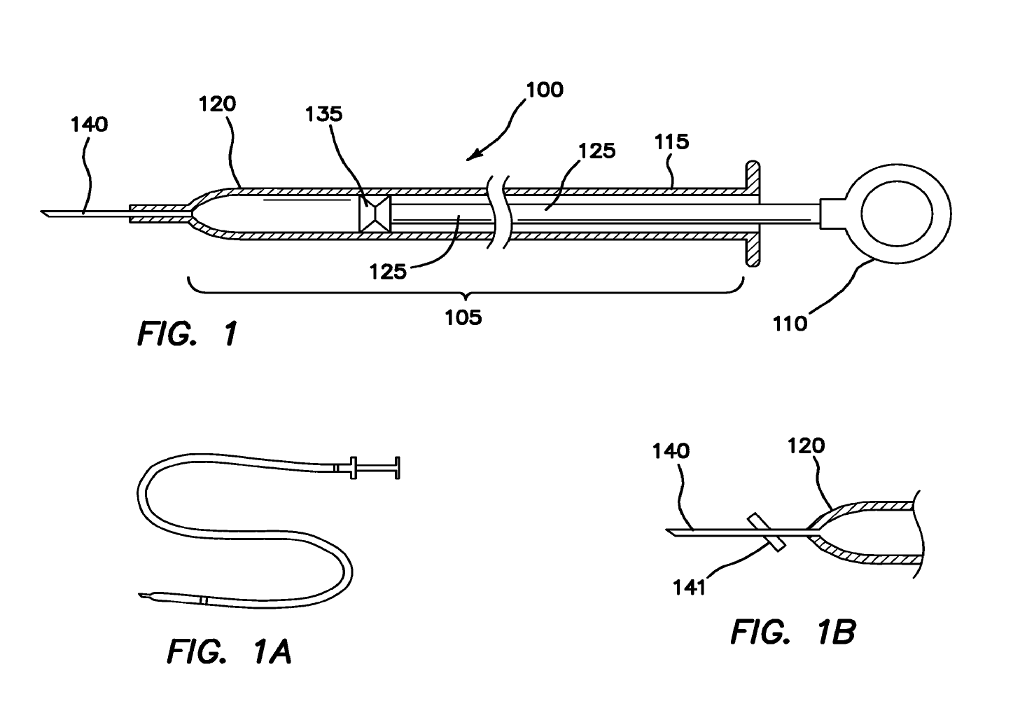

Referring more particularly to the drawings, FIG. 1 is a simplified cross-sectional drawing of a portion of an injection apparatus 100 (e.g., a syringe) comprising an elongated body 105 (e.g., catheter) having disposed therein a movable stopper or piston 135. The piston 135 may separate the body 105 of the syringe 100 into a first body portion 115 disposed proximally to the piston 135 and a second body portion (e.g., chamber) 120 disposed distally to the piston 135, where it is understood that, as used herein, the term “proximal” means an end or part nearest to an operator of an instrument (e.g., the injection apparatus 100). Conversely, the term “distal” refers to an end or part furthest from the operator. All figures presented herein are oriented with the proximal portions located to the right of distal portions, which, generally, are on the left. In this disclosure, the proximal end of an object may be referred to as the first end, and the distal end of the object may be referred to as the second end.

In the exemplary embodiment illustrated in FIG. 1, body 105 of the syringe is relatively long and contiguous. In representative embodiments, the length of the body 105 may range from about 10 cm to about 140 cm. The movable piston 135 may have a plunger wire 125 affixed to a proximal side thereof, the plunger wire 125 extending through a lumen of the first body portion 115 and terminating at, for example, a ring 110. The ring 110, which may be affixed to a proximal end of the wire 125, may be adapted to fit and be operated by, for example, a finger (e.g., thumb) of an operator. The ring 110 may provide a means to control the piston using the wire. In particular, a pushing or pulling force applied to the ring 110 may be transmitted by the wire 125 to the piston 135.

A distal end of the second body portion 120 may terminate in a hollow distal needle 140, the needle 140 being adapted to receive material from an interior of the second body portion 120 distal to the piston 135 and to administer the material to a patient when the needle 140 is inserted into the body of the patient. In a representative application, the second body portion 120 may also be adapted to receive therapeutic material through the needle 140. The therapeutic material, examples of which may include a relatively high-viscosity material such as collagen, may be drawn into the second body portion 120 through the needle 140 by applying a pulling force to the ring 110, which pulling force may displace the piston 135 proximally, thereby drawing in the therapeutic material. Other examples of a therapeutic material may include a medium (e.g., collagen) of microspheres, as is disclosed in U.S. Pat. No. 5,344,452, the contents of which are expressly incorporated herein by reference in their entirety with respect to the methods and/or structures described therein.

FIG. 1A is a cross-sectional drawing of a portion of a long injection device employing a plunger wire adapted for activating a movable stopper or piston, and FIG. 1B illustrates a crest or tissue stop 141 disposed to surround the distal needle 140 near the distal end of the second body portion 120. The tissue stop 141 may be sufficiently large to control a depth of injection of the distal needle 140 into the body of a patient.

In particular, in certain surgical procedures, e.g., esophageal sphincter bulking, it is critical to the success of the procedures that injections of therapeutic material, e.g., microspheres, be submucosal and not intramuscular, particularly if the therapeutic material is to be removed at some time after initial treatment. Failure to observe the submucosal restriction may result in perforation of the esophageal wall and may place the therapeutic material outside of the esophagus into the mediastinum, aorta or even the heart. Indeed, known prior-art therapeutic material was overly viscous to an extent that an 18G needle was required to inject it, rendering it relatively unplaceable submucosally (meaning it could not be placed strictly submucosally) and subject to being injected into the muscle resulting in perforation of the esophageal wall (e.g., physicians perforating the esophageal wall and ending-up injecting it into the mediastinum (around the heart) and even into the aorta) with potentially fatal results. As a consequence of the entire esophageal wall (mucosa, muscle, serosa) being only about 4-5 mm thick, with the mucosa typically being only 0.5 μm thick, the needle tip of the invention is designed and operated so as imperatively and reliably not to penetrate into the muscle to avoid any intramuscular injections as those cannot be removed, if need be, without damaging the muscle. Additionally, the prior-art material was not sufficiently tissue biocompatible and prone to being sloughed off. The therapeutic material contemplated by the present invention, i.e., microspheres, on the other hand, has fewer to none of these shortcomings when injection is performed correctly through a 23G needle placed strictly submucosally. (A mucosal “bleb has to rise” under direct visualization until esophageal walls are adapted and lumen is closed.) Accordingly, in an esophageal bulking procedure, the tissue stop 141 is preferably disposed at a distance of about 2 mm from the tip of the needle 140. While an expanded esophagus wall has a thickness of about 2 mm, a relaxed esophagus may have a thickness of about 4-5 mm. The esophageal bulking procedure may employ endoscope, which may expand the esophageal wall. In this instance, use of a 2 min tissue stop 141 assures that the therapeutic material is injected strictly into the submucosal plane. If the muscle is not perforated with the needle 140, then the therapeutic material may be “milked” towards the submucosal space and not to the outside the esophagus (subserosally).

One embodiment of the tissue stop 141 comprises a polymeric material having a circular perimeter, but which may be oval, rectangular, or of another shape in alternative embodiments. In another embodiment, the tissue stop 141 comprises stainless steel.

An angle between a plane of the tissue stop 141 and a longitudinal axis of the distal needle 140 is preferably less than ninety degrees and, (preferably, less than about seventy-five degrees and, more preferably, about sixty degrees as shown in FIG. 1. The orientation of the tissue stop 141 may be selected no that a planar surface of the tissue stop 141 will align longitudinally with the axis of a particular lumen that is being treated. In other words, a planar surface of the tissue stop 141 should rest flat on the surface of the tissue that is to be treated with the distal needle 140. The tissue stop 141 may help to prevent the needle from penetrating deeper into the tissue than is required, permittable, optimal, or desired.

A surgeon performing an injection procedure using, for example, a cystoscope or the device disclosed in U.S. patent application Ser. No. 09/825,484, entitled URETHRA SURGICAL DEVICE, can view the tissue stop 141 for assistance in performing an injection at a proper angle and at a proper depth.

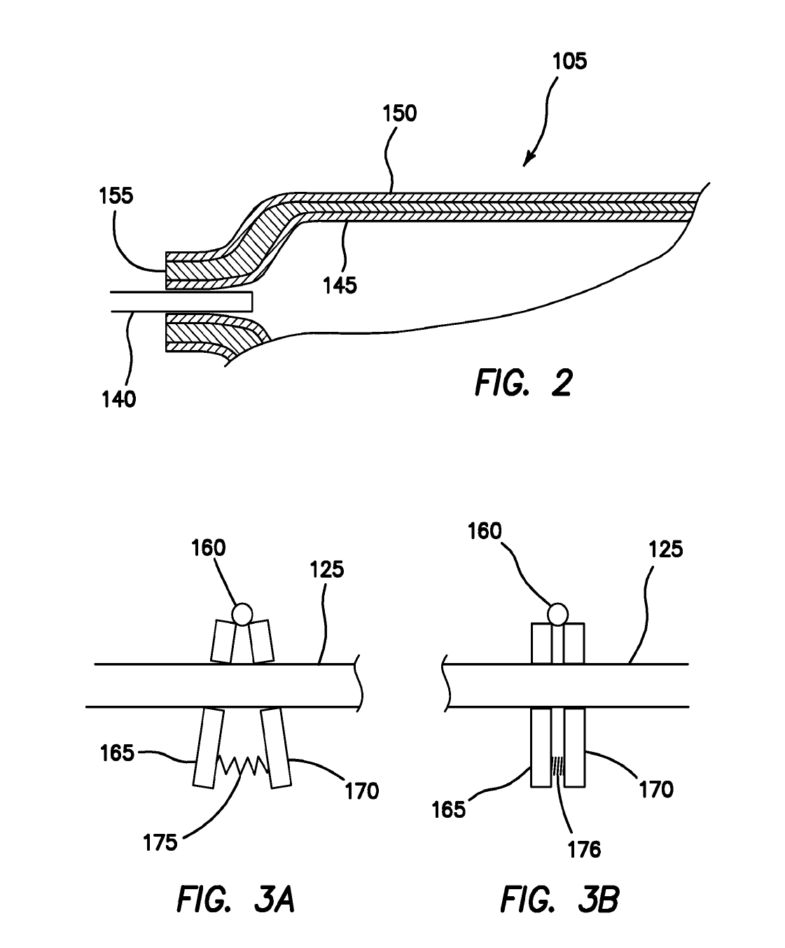

According to a feature of the catheter device, tractability is very important. That is, the elongated syringe must be able to navigate a tortuous path to get to where treatment is needed. One embodiment of the present invention includes the use of a co-extrusion for the body of the syringe. Layers of different material can be fabricated during the manufacturing process of the tube for which the body is made. For example, FIG. 2 is a partial cut-away diagram of a cross-sectional view of the body 105 of the syringe illustrated in FIG. 1 describing one particular implementation of the body 105. In the illustrated embodiment, the body 105 is formed as a co-extrusion into a plurality of layers. Three layers are shown in FIG. 2: a thin, lubricous inner layer 145, formed of, for example, Teflon® or similar polymer that, together with the piston 135 can provide a seal that separates the first body portion 115 and the second body portion 120. The body 105 further comprises a hydrophilic thin lubricous outer layer 150 capable of passing through a luminal structure. A middle strength layer 155 may be thicker than inner and outer layers 145 and 155 and may be formed of a linear low-density polyethylene having good whoop strength but lateral pliability for negotiating a tortuous path.

The device illustrated in FIGS. 1 and 2 may be minimally invasive, such as in the context of being normally used through a natural opening in the body or through a cannula designed to being accepted into various lumens such as of the vascular system. It is contemplated that this device be used to augment heart valves or to repair any structure that can be reached with a small diameter catheter. (The terms sphincter and valve are used interchangeably in this disclosure.) The device, further, may be adaptable to treatment of urinary incontinence as described more fully in the '848 patent while also being preferentially adaptable to the treatment of GERD and fecal incontinence, where a bulking agent is injected into the submucosal space around the internal anal sphincter muscle. Treatment of a urinary incontinence or fecal incontinence condition may comprise the injection of a filler material, such as collagen, into and adjacent to the urinary sphincter muscle at the bladder neck or the internal anal sphincter muscle, to thereby bulk up the tissue and assist in the adequate closure of the urinary sphincter. Filler material injection may be employed in the treatment of GERD as disclosed herein, which injection may be an effective alternative to Nissen fundoplication and other surgical procedures. In all cases, it is important not to interfere with normal sphincter functions.

In certain applications, such as in GERD treatment, a relatively long stroke on, for example, the ring 110 of FIG. 1 may be required to draw-up a typical dose of about 2 cc of fluid for injection into a patient. The present invention contemplates the use of several different mechanisms to accommodate the necessary travel of the wire 125 (FIG. 1). One such mechanism, illustrated schematically in FIGS. 3A and 3B, is a crab washer mechanism used, for example, on commercially available caulking gun products found in a hardware store. FIG. 3A illustrates, in cross-section, a crab washer mechanism in a locked state. Two washers 165 and 170, hingeably attached to a pin 160, may encircle the wire 125 and may be forced apart by a spring 175. The illustrated state permits motion of the wire 125 only in the leftward direction as will be evident to one skilled in the art. Conversely, as illustrated in FIG. 3B, in an unlocked state, the washers 165 and 170 may be pressed together (e.g., manually, or using another mechanism not shown in opposition to force of the spring 176, thereby allowing the wire 125 to move in either direction. Such a mechanism can be used to push over long distances as contemplated by the present invention.

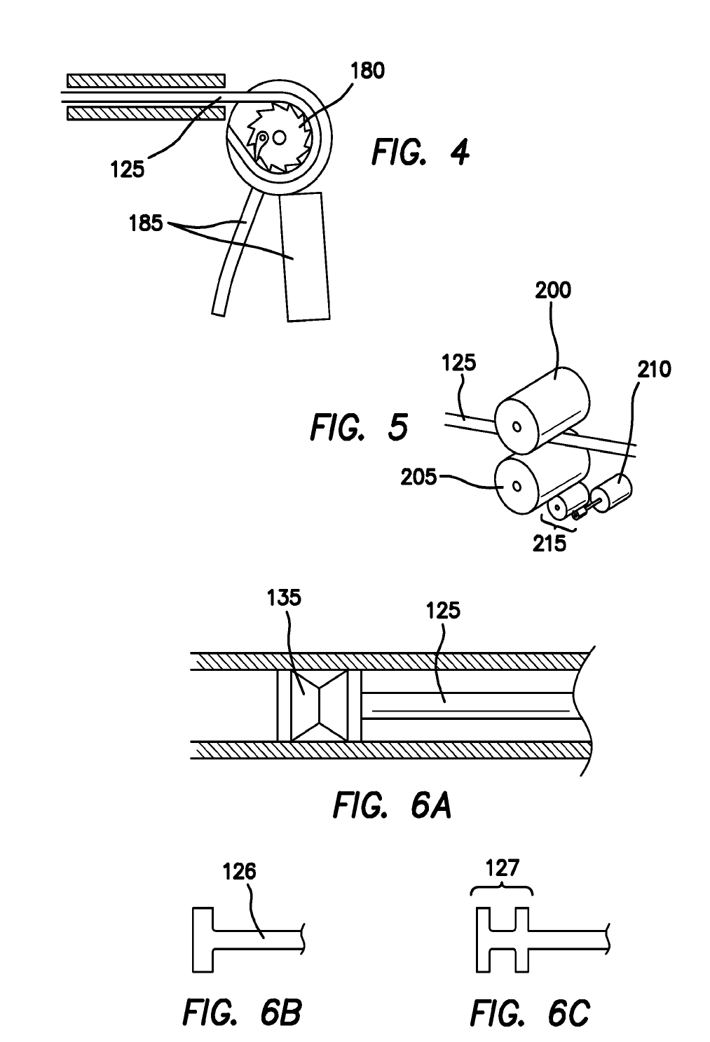

Another mechanism that may facilitate a long stroke on the wire 125 of FIG. 1 may comprise a spool that stores extra length of the plunger wire 125. FIG. 4 is a partial cross-sectional schematic diagram of such a spool that includes a ratchet mechanism operated by gripping a pair of handles. In use, the mechanism of FIG. 4 may be combined with the mechanism of FIGS. 3A and 3B to advance the wire 125 in small steps, as may be required in some medical procedures.

Yet another mechanism to control a position of the wire 125 (FIG. 1) comprises a pair of rollers with the wire 125 disposed between them. As illustrated in FIG. 5, an upper roller 200 and a lower roller 205 may grip the wire 125. Motion of the wire 125 may be controlled by rotating the rollers 200 and 205 using, for example, a stepper motor 210 that drives one of the rollers 200 or 205 through a gear reduction set 215. Controlling the stepper motor with an electronic circuit and firmware may provide very precise motion of the wire 125 in order that the amount of fluid administered to a patient can be accurately controlled. Also, the rate of injection can be controlled. So if there is a need to inject fluid over a set amount of time, this can be programmed. In most cases where the mucosa is healthy enough to hold the bulking agent, the desired bulking of a sphincter or other structures can be reached as fast or as slow as the clinician desires. Certain anatomic structures and specifically inflamed tissues are more sensitive to the rate at which the tissue is displaced, such as in the case of esophagitis (inflamed mucosa) or Barrett's esophagus (advanced, pre-cancerous condition). For instance, during an injection bulking procedure, the esophageal, urinary and/or anal sphincter mucosa may be separated slowly enough from the underlying muscle so that disruption of blood and lymphatic vessels can be minimized or avoided when/with a fluid or bulking agent is/being injected at adjustable (e.g., relatively slow) rates. This is particularly useful when there exists a lesion or friable tissue in the vicinity. This may be very desirable when using a bulking method to cause particular desired results such as ischemic therapy for cancerous lesions. Such double roller systems are regularly used for arterial visualization when a constant rate of velocity is required. In particular, intravascular ultrasound (IVUS) and optical coherence tomography (OCT) angiography require a constant rate of velocity.

Attachment of the piston 135 to the wire 125 (FIG. 1) is illustrated in FIGS. 6A, 6B, and 6C. The piston 135, shown in FIG. 6A, may be insert molded over the distal end of the wire 125 and may be created from a low-friction polymer such as Teflon®. The distal end of the wire 125 may be cold formed to create a very good attachment to the piston. To facilitate the attachment, the distal end of the wire 125 may be expanded with a single head 126 as shown in FIG. 6B or with a plurality of heads, e.g., a double head 127 (FIG. 6C), and the polymer piston 135 may be molded on the single or double head 126 or 127. According to one implementation, the distal end of the wire 125 comprises a male threaded portion that screws into a corresponding female threaded portion disposed on a proximal side of the piston, thereby facilitating convenient attachment/removal.

### Additional Features and Blocking:

1) Photoluminescent needle tip and possible Length Markers (also photoluminescent optionally) to help determine or better verify needle insertion point and insertion depths and length of delivered bleb beads while inserting and withdrawing needle.
2) Use of catheter materials such as PBAX to provide Ultra Low coefficients of friction for lowest possible extrusion force requirements (along with good tensile and hoop strength per cross sectional wall area).
3) Lubricious and or hydrophilic coating options for catheter (and needle) inner walls to minimize capillary restriction and allow for reduced extrusion force requirements.
4) Remotely retracting, extending, or uncovering needle tip to guard against unwanted perforation of adjacent anatomical structures during needle feeding and placement.
5) Mechanically multiplied injection assist (such as the ratcheting pistol or cam activated force multiplier) to reduce thumbpad extrusion force requirements and improve injection control.
6) Digitally controlled surgeon activated switch (e.g., button on the handle, or hand activated, or foot pedal) activated pneumatic assistance to the syringe plunger to provide (one) hands five assistance to the syringe plunger, while also giving the surgeon control over the exact amounts of bulking agent delivered to each bleb strand.
7) Length markers or indexing mechanism on catheter and/or needle to help lock needle to same position on gastroscope.
8) Malleable wire extruded into catheter tubing wall to provide rigidity and ability to produce an angle of incidence for catheter and needle to facilitate observation of injections into the esophageal wall.
9) Optional Purging Rod or fluid media purging to minimize product holdup loss in the catheter.
10) Pre Adjustable Stop on outside needle shaft to allow preset depth control. Can be set with clip or set screw or be factory pre-set with bonding. Stop flange can also be angled if advantageous.
11) Syringe barrel to be constructed of ultra high moisture barrier transparent material, such as non-leaching glass, or a Cyclic Olefin Copolymer such as Topas, or Zenex, or, Zylar, or layered laminate thereof, combined with high barrier plunger tip and closure to maintain product integrity in storage.
12) Straight lathe machined stainless tube injection needle with beveled and mildly radiused transition bore area to provide very cost effective and minimally restrictive flow for low plunger forces and maintenance of uniform microsphere distribution (see profile sketch on next page). Use UV light curing adhesive or Raumedic bonding of catheter to stainless steel needle shaft with textured outer wall.

### Additional Description:

An injection device for G125 attached or attachable (via Luer lock) to a long catheter is provided that can be pushed through a standard working channel of a standard sigmoidoscope or gastroscope and which allows for precise and strictly ‘submucosal’ injections of a viscous bulking agent. The needle at the end of the catheter is constructed to be small enough to enter this submucosal space (e.g. 23G) yet large enough to allow for an injection of millions of 125 micron PMMA microspheres suspended in a viscous carrier medium such as for example collagen or hyaloronic acid (but not limited to those materials).

It is of utmost importance that the injection ‘pressure’ through such a 40 inch (estimate) long catheter is acceptable for even female injectors and that the needle will not clog. The needle can preferably have a stopper at 2 mm from the needle tip to avoid too deep (‘intramuscular’) placement of the soft tissue bulking agent. It is important or preferred that the injection is performed under ‘direct visualization’ so that the injector can see the actual ‘mucosal bleb’ rise during injection to assure the correct plane of injection and also to determine the correct injection volume until the esophageal mucosa is completely adapted (360 degrees). Injections of 3 blebs in a circumferential pattern can be implemented to achieve complete adaptation (similar to a tricuspid aortic valve with no opening in the center—see image in the below excerpted white paper). The opposing blebs can either be injected on the same level or on different, slightly offset longitudinal levels to achieve an even better barrier towards acid reflux.

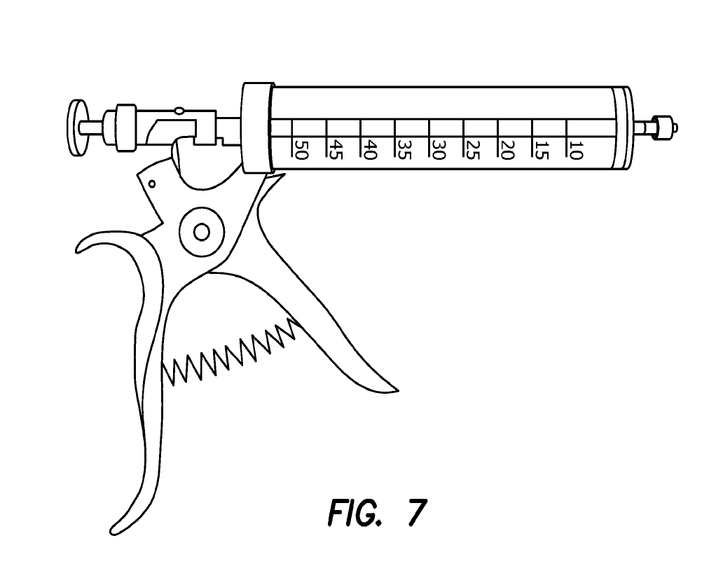

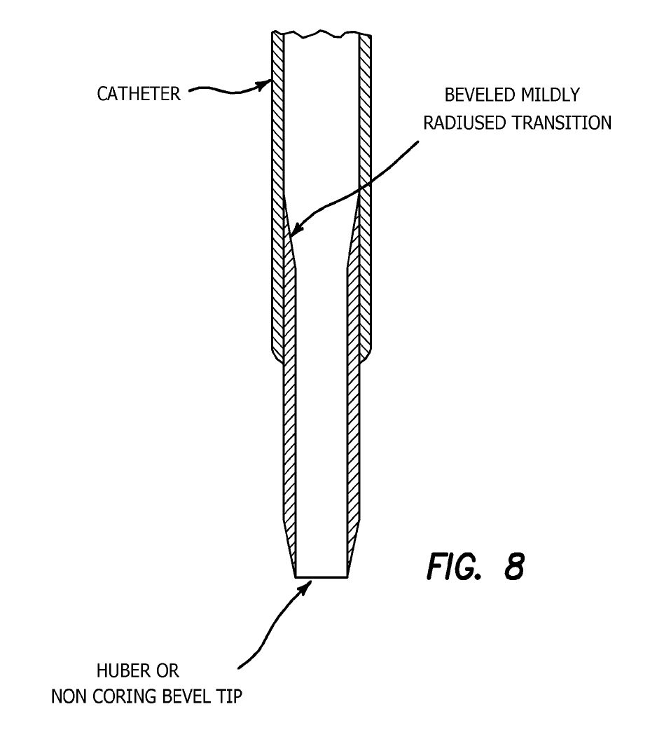

An aspect of the present invention is user-friendliness whereby a gastroenterologist or surgeon who has placed the scope down the esophagus to inspect the severity and damage caused by GERD is able to easily push the inventive catheter through a working channel and start injecting G125 without removing the scope and catheter until the G125 procedure is completed. According to one aspect, the injection device is able to accommodate a standard syringe. A preferred fill volume per syringe is 2 cc. According to one feature, an assembly of 3 syringes along with one catheter per G125 ‘unit’ is provided as a kit thereby facilitating an ability or convenience of leaving the catheter in place and only change the syringes. According to another embodiment of the present invention, a vial (e.g., a 10 ml vial) is affixed to a pistol mechanism as illustrated in FIG. 7. The vial and pistol mechanism may attach at the proximal end of the long injection device of FIG. 1A, thereby eliminating the need for a wire 125 and piston 135 with an advantage that large amounts of therapeutic material may be injected without removing the needle 140 from the injection site. This arrangement may permit the flow of high-viscosity collagen through the long injection device while using, e.g., a 230 or 210 needle 140. In accordance with yet another embodiment of the present invention, as illustrated FIG. 8, a low cost needle with minimal flow losses is provided with a construction having virtually no ridge(s) that if present would allow microspheres to collect resulting in a disruption of the homogeneity. The distal most end of the injection cylinder can, according to one implementation, have ribs that let the G125 spheres file one at a time into the proximal end of the needle. The architecture of ribs can be characterized, according to one embodiment, as similar to an Urbanti Funnel (which has internal helicoid ribs to increase the filtration speed in that device). For example, the helicoid ribs can serve as a staging area for the spheres to line up before they enter the proximal most end of the needle. This may help to address any problem of the spheres losing any velocity they had, which is necessary to avoid a “log jam” just before they feed into the needle. The Urbanti Funnel can address a valuable need in this context and/or when combined with the movable piston optionally being formed to have a complementary surface in order to inject the maximum possible viscous fluid with suspended spheres.

The present invention may be practiced in accordance with methods and materials described or referenced in “Endoscopic lower esophageal sphincter bulking for the treatment of GERD: safety evaluation of injectable polymethylmethacrylate microspheres in miniature swine” authored by Jan P, Kander, MD, Gottfried Lemperle, MD, PhD, Stefan Lemperle, MD and Glen A. Lehman, MD (Gastrointest Enclose. 2010 August; 72(2):337-42. Epub 2010 Jun. 11. PMID: 20541193 [PubMed—indexed for MEDLINE]; cf. www.giejournal.org) and “Urethral Bulking With Polymethylmethacrylate Microspheres for Stress Urinary Incontinence: Tissue Persistence and Safety Studies in Miniswine” authored by Gottfried Lemperle, Patrick B. Lappin, Corbett Stone and Stefan M. Lemperle (Urology. 2011 April; 77(4): 1005.e1-7. Epub 2011 Feb. 18; PMID: 21333337 [PubMed—in process]; cf. doi: 11.0.1016/j.urology.2011.12.021), the contents both of which are incorporated herein by reference. According to one implementation or aspect, a minimum 125 micron PMMA microsphere size is established as being safe to avoid intravascular or lymphatic transportation during esophageal and urinary submucosal injections. According to another, the 125 micron PMMA microsphere size is a crucial ‘minimum microsphere size’ for one or more of (a) GERD, (b) SUL (c) FI and/or (d) to avoid migration through lymphatic and blood vessels. In yet another, about 100 to 150 micron microspheres may be determined as safe for use internally in sphincters an/or a preferred needle size is 21-25G, whereas smaller microspheres (30-50 micron) may be determined for use subdermally. The present invention further may be practiced in accordance with methods and materials described or referenced in “A New, Permanent Injectable Bulking Agent for the Endoscopic Treatment of Heartburn (GERD)” authored by Gottfried Lemperle, MD, PhD and Stefan M. Lemperle, MD, the contents of which are incorporated herein by reference and an excerpt of which follows,

In view of the foregoing, it will be understood by those skilled in the art that the methods and devices of the present invention can facilitate formation of injection apparatuses. The above-described embodiments have been provided by way of example, and the present invention is not limited to these examples. Multiple variations and modification to the disclosed embodiments will occur, to the extent not mutually exclusive, to those skilled in the art upon consideration of the foregoing description. For example, body portions may have cross-sections that are substantially circular, elliptical, rectangular, or the like, or that take other types of shapes altogether. Bevels and/or chamfers, for example, may be introduced as disclosed, for example, in the above-referenced '848 patent. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. A long injection device, comprising:
an elongated body having disposed therein a movable piston having a distal side and a proximal side, wherein the piston divides the body into a first portion proximal to the piston and a second portion distal to the piston;
a wire having a distal end attached to the proximal side of the piston, the wire extending to a proximal end of the first body portion and being controllable from the proximal end of the first body portion, whereby the piston is capable of being moved by the wire within the elongated body; and
a hollow distal needle disposed at a end of the second body portion, whereby, when the distal needle is inserted into the body of a patient, motion of the piston is capable of causing material disposed in an interior of the second body portion to be injected into the body of the patient.

2. The long injection device as set forth in claim 1, further comprising a tissue stop disposed around the distal needle so that the distal needle is inserted submucosally.
